(19) United States
(12) Patent Application Publication
(10) Pub. No.: US 2024/0315607 A1
(43) Pub. Date: Sep. 26, 2024

(54) METHODS AND SYSTEMS FOR IMPROVED SURGICAL CARE

(71) Applicant: Know Labs, Inc., Seattle, WA (US)

(72) Inventor: John CRONIN, Williston, VT (US)

(21) Appl. No.: 18/188,000

(22) Filed: Mar. 22, 2023

(51) Int. Cl. A61B 5/145; G16H 40/63

(52) U.S. Cl. CPC A61B 5/14532; A61B 5/14546; G16H 40/63

(57) ABSTRACT

Systems and methods for improved surgical care of a patient in which real-time radio frequency glucose readings and real-time radio frequency non-glucose analyte readings are obtained during the surgical care of the patient, displayed to the caregiver in real-time, and used to inform the caregiver in real-time.

### FIELD

The present disclosure is generally related to improve surgical care with a real-time analyte monitoring device.

### BACKGROUND

Currently, medical monitoring devices are not able to collect and inform medical professionals with a patient's analyte data in real-time, such as glucose levels. Also, medical monitoring devices are not able to analyze a patient's physiological parameters that include analyte levels to determine recommendations to inform the medical professionals. Lastly, medical monitoring devices cannot provide actionable recommendations that are derived from analyzing a patient's analyte levels with other physiological parameters. Thus, there is a need in the prior art to provide a method for improved surgical care using a real-time analyte monitoring device.

### DESCRIPTION OF THE DRAWINGS

FIG. 1: Illustrates a system for improved surgical care, according to an embodiment.

FIG. 2: Illustrates an example operation of a Base Module, according to an embodiment.

FIG. 3: Illustrates an example operation of a Glucose Module, according to an embodiment.

FIG. 4: Illustrates an example operation of an Analyte Module, according to an embodiment.

FIG. 5: Illustrates an example operation of a Feedback Module, according to an embodiment.

FIG. 6: Illustrates an example operation of a Display Module, according to an embodiment.

FIG. 7: Illustrates an Analyte Database, according to an embodiment.

FIG. 8: Illustrates a Feedback Database, according to an embodiment.

FIG. 9: Illustrates a Second Embodiment of a system for improved surgical care.

FIG. 10: Illustrates a Third Embodiment of a system for improved surgical care.

FIG. 11: Illustrates a Fourth Embodiment of a system for improved surgical care.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety. U.S. 2022/0192522 is also incorporated herein by reference in its entirety.

As used herein and in the claims, the term “surgical care” includes care of a patient during a surgical procedure on the patient, as well as pre-operative care of the patient and post-operative care of the patient. The surgical care may take place in an operating room or in any other room where the patient may be located during surgical care.

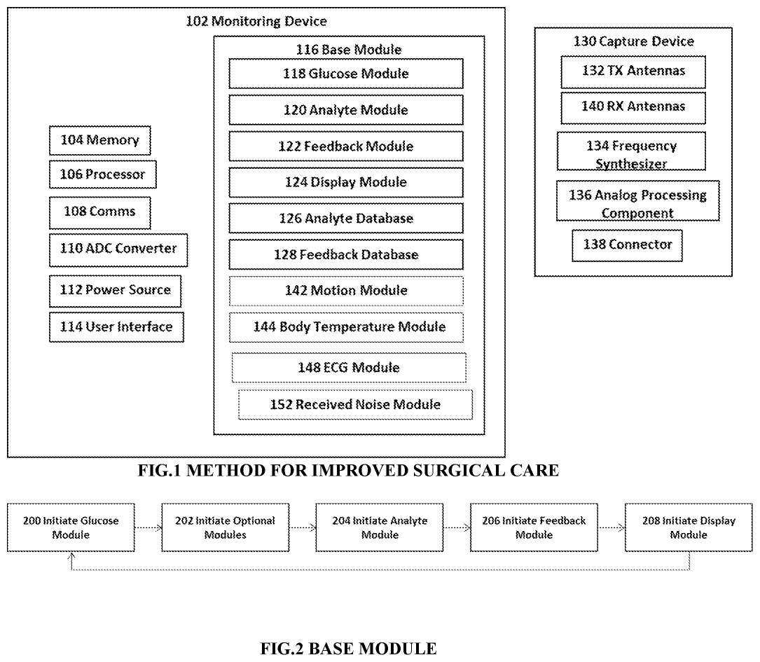

FIG. 1 illustrates a method for improved surgical care. This method comprises an analyte monitoring device 102 that may be used to measure, detect, and display physiological parameters of a patient to inform medical professionals of the physiological parameters. The analyte monitoring device 102 may receive data from a connector 138 to display the data collected from the capture device 130. The analyte monitoring device 102 may include a memory 104, a processor 106, comms 108 such as physical connections to the plurality of devices, ADC converter 110, a power source 112, a user interface 114, and a base module 116 that may be continuously running and initiating the glucose module 118, the analyte module 120, the feedback module 122, the display module 124, the motion module 142, the body temperature module 144, the ECG module 148, and the received noise module 152. The monitoring device 102 may store, extract, compare, etc. data from the analyte database 126 and the feedback database 128. Further, embodiments may include the memory 104 that may be configured to store the transmitted Activated RF range signals by the one or more TX antennas 132 and receive a responded portion of the transmitted Activated RF range signals from the one or more RX antennas 140. Further, the memory 104 may also store the converted digital processor readable format by the ADC converter 110. In one embodiment, the memory 104 may include suitable logic, circuitry, and/or interfaces that may be configured to store a machine code and/or a computer program with at least one code section executable by the processor 106. Examples of implementation of the memory 104 may include, but are not limited to, Random Access Memory (RAM), Read Only Memory (ROM), Hard Disk Drive (HDD), and/or a Secure Digital (SD) card.

Further, embodiments may include a processor 106 which may facilitate the operation of the analyte monitoring device 102 to perform functions according to the instructions stored in the memory 104. In one embodiment, the processor 106 may include suitable logic, circuitry, interfaces, and/or code that may be configured to execute a set of instructions stored in the memory 104. The processor 106 may be configured to run the instructions obtained by the monitoring device base module 116 to perform analyte monitoring. The processor 106 may be further configured to collect real-time signals from the one or more TX antennas 132 and the one or more RX antennas 140 and may store the real-time signals in the memory 104. In one embodiment, the real-time signals may be assigned as initial and updated radio frequency (RF) signals. Examples of the processor 106 may be an X86-based processor, a Reduced Instruction Set Computing (RISC) processor, an Application-Specific Integrated Circuit (ASIC) processor, a Complex Instruction Set Computing (CISC) processor, and/or other processors. The processor 106 may be a multicore microcontroller specifically designed to carry multiple operations based upon pre-defined algorithm patterns to achieve the desired result. Further, the processor 106 may take inputs from the analyte monitoring device 102 and retain control by sending signals to different parts of the capture device 130. The processor 106 may access a Random Access Memory (RAM) that is used to store data and other results created when the processor 106 is at work. It can be noted that the data is stored temporarily for further processing, such as filtering, correlation, correction, and adjustment. Moreover, the processor 106 carries out special tasks as programs that are pre-stored in a Read Only Memory (ROM). It can be noted that the special tasks carried out by the processor 106 indicate and apply certain actions which trigger specific responses.

Further, the comms 108 of the device 102 may communicate with the capture device 130 through a connector 138. Examples of the comms 108 may include, but are not limited to, a physical connection, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN). In one embodiment, various devices may be configured to have a communication module integrated over circuitry arrangement to connect with the device network via various wired and wireless communication protocols, such as the cloud network. Examples of such wired and wireless communication protocols may include, but are not limited to, Transmission Control Protocol and Internet Protocol (TCP/IP), User Datagram Protocol (UDP), Hypertext Transfer Protocol (HTTP), File Transfer Protocol (FTP), Zigbee, EDGE, infrared (IR), IEEE® 802.11, 802.16, cellular communication protocols, and/or Bluetooth® (BT) communication protocols.

Further, embodiments may include the ADC converter 110 which may be coupled to the one or more RX antennas 140. The one or more RX antennas 140 may be configured to receive the responded Activated RF range signals. The ADC converter 110 may be configured to convert the received Activated RF range signals from an analog signal into a digital processor readable format.

Further, embodiments may include a power source 112 which is a source of electrical energy, such as a battery or power line. Further, embodiments may include a user interface 114 which may either accept inputs from users or provide outputs to the users, or may perform both the actions. In one case, a user can interact with the interface(s) using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or a combination of the above. Further, the interface(s) may either be implemented as a Command Line Interface (CLI), a Graphical User Interface (GUI), a voice interface, or a web-based user-interface.

Further, embodiments may include the base module 116 which initiates the glucose module 118, the analyte module 120, the feedback module 122, and the display module 124. Further, embodiments may include the glucose module 118 which begins by being initiated by the base module 116. The glucose module 118 sends the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. For example, the one or more TX antennas 132 transmit an Activated RF range signal between 500 MHz and 300 GHz. The glucose module 118 stores the RF transmit signal to memory 104. The glucose module 118 receives the RF signal from the capture device 130 RX antenna 140. For example, the one or more RX antennas 140 may be configured to receive the responded portion of the Activated RF range signals. In one embodiment, the Activated RF range signals may be transmitted into the user, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted Activated RF range signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 140. The glucose module 118 converts to digital using the ADC converter 110. The glucose module 118 stores the RX converted signal data in memory 104. The glucose module 118 correlates the RF signals with ground truth data to determine the glucose numbers. The glucose module 118 stores the glucose number in the analyte database 126.

Further, embodiments may include an analyte module 120 which begins by being initiated by the base module 116. The analyte module 120 sends the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. The analyte module 120 stores the RF transmit signal to memory 104. For example, the glucose module 118 stores the transmitted signal to memory 104. The analyte module 120 receives the RF signal from the capture device 130 RX antenna 140. For example, the one or more RX antennas 140 may be configured to receive the responded portion of the Activated RF range signals. In one embodiment, the Activated RF range signals may be transmitted into the user, and electromagnetic energy may be responded from many parts such as fibrous tissue, muscle, tendons, bones, and the skin. It can be noted that effective monitoring of the analyte level is facilitated by an electrical response of blood molecules against the transmitted Activated RF range. Further, the electromagnetic energy responded from the blood molecules may be received by the one or more RX antennas 140. The analyte module 120 converts to digital using the ADC converter 110. The analyte module 120 stores the RX converted signal data in memory 104. The analyte module 120 correlates the RF signals with ground truth data to determine the analyte numbers. The analyte module 120 stores the analyte levels or oxygen saturation levels in the analyte database 126.

Further, embodiments may include a feedback module 122 which begins by being initiated by the base module 116. The feedback module 122 extracts the data entry from the analyte database 126. The feedback module 122 compares the extracted data entry from the analyte database 126 to the feedback database 128. The feedback module 122 determines if there is a recommendation. If it is determined that there is a recommendation the feedback module 122 extracts the recommendation from the feedback database 128. The feedback module 122 sends the extracted recommendation from the feedback database 128 to the display module 124. If it is determined that there is no recommendation or after the recommendation has been sent to the display module 124 the feedback module 122 returns to the base module 116.

Further, embodiments may include a display module 124 which begins by being initiated by the base module 116. The display module 124 extracts the data entry from the analyte database 126. The display module 124 displays the data entry on the user interface 114. The display module 124 determines if a recommendation was received from the feedback module 122. If it is determined that there was a recommendation received from the fuse module 122 the display module 124 receives the recommendation from the feedback module 122. The display module 124 displays the received recommendation on the user interface 114. If it is determined that there was no recommendation received from the feedback module 122 or after the recommendation is displayed on the user interface 114 the display module 124 returns to the base module 116.

Further, embodiments may include an analyte database 126 which is created from the process described in the glucose module 118 and analyte module 120 which collects data from the capture device 130. The data is compared to the feedback database 128 to determine if there is a relationship between the data that requires the medical professional's attention and provides the medical professional with a notification. The data is extracted from database and displayed on the analyte monitoring device 102 user interface 114 to inform the medical professional of the patient's physiological parameters. The database contains a patient ID, the time in which the data was collected and displayed on the analyte monitoring device 102, the patient's glucose numbers, and the patient's oxygen saturation levels. In some embodiments, the database may include additional analyte data, such as lactate, cholesterol, etc.

Further, embodiments may include a feedback database 128 which contains a predetermined set of rules that provide a medical professional with a notification or alert on the analyte monitoring device 102 if the patient's physiological parameters are at certain levels or exceed certain thresholds that may cause harm to the patient. The database is used in the process described in the feedback module 122 in which the data from the analyte database 126 is compared to the thresholds provided in the feedback database 128 to determine if the patient's physiological parameters exceed one, two, or a combination of the thresholds and, if so, the rule or recommendation is extracted and is displayed on the analyte monitoring device 102 user interface 114. The database may contain thresholds for the patient's glucose numbers, oxygen saturation levels, and the corresponding rule. In some embodiments, the database may include additional analyte data, such as lactate, cholesterol, etc.

Further, the capture device 130 may be connected to the analyte monitoring device 102 through a connector 138. In one embodiment, the connector 138 may be a wireless and/or wired communication channel. The capture device 130 may be worn by the user. The capture device 130 may determine health parameters using radio frequency signals in the Activated RF range. In one embodiment, the health parameters may include blood sugar or blood glucose levels, oxygen saturation levels, lactate, cholesterol, other analytes, etc. The system may target specific blood molecules using the Activated RF range signals, and output signals from the molecules may correspond to the analyte levels in the user.

In one embodiment, the system may include integrated circuit (IC) devices (not shown) with transmit and/or receive antennas integrated therein. For example, monitoring the blood glucose level of the user using the Activated RF range involves the transmission of suitable Activated RF range signals into the user. Corresponding to the transmission, a responded portion of the Activated RF range signals is received on multiple receive antennas. Further, the system isolates and/or processes a signal in response to the received Activated RF range signals. The system may output a signal from the received Activated RF range signals that correspond to the blood glucose level in the user. It can be noted that the capture device 130 may be worn by the user at various locations such as wrist, arm, leg, etc. In one embodiment, the system for monitoring the analyte levels of the user using the Activated RF range signals involves transmitting Activated RF range signals into a user, receiving a responded portion of the Activated RF range signals on multiple receive antennas, isolating a signal from the Activated RF range signals in response to the received Activated RF range signals, and outputting a signal that corresponds to the analyte levels in the user in response to the isolated signal. In one embodiment, beamforming is used in the receiving process to isolate the Activated RF range signals responded from a specific location of the user to provide a high-quality signal corresponding to the analyte levels in the user. In another embodiment, Doppler effect processing may be used to isolate the Activated RF range signals responded from the specific location to provide the high-quality signal corresponding to the analyte levels in the user. It can be noted that analog and/or digital signal processing techniques may be used to implement beamforming and/or Doppler effect processing and digital signal processing of the received signals to dynamically adjust a transmitted beam onto the desired location. In another embodiment, the beamforming and the Doppler effect processing may be used together to isolate the Activated RF range signals responded from the user to provide the high-quality signal corresponding to the analyte levels in the user. For example, in one exemplary embodiment, Activated RF range signals of a higher frequency range of 122-126 gigahertz (GHz) having a shallower penetration depth are used to monitor blood glucose levels. It can be noted that the shallower penetration depth reduces undesirable reflections, such as reflections from bone and dense tissue such as tendons, ligaments, and muscle, which may reduce the signal processing burden and improve the quality of the desired signal that is generated from the user. It can also be noted that bones are dielectric and semi-conductive. In addition, bones are anisotropic, so not only are bones conductive, but they also conduct differently depending on the direction of the flow of current through the bone. Alternatively, the bones are also piezoelectric materials. Therefore, an Activated RF range signals of higher frequency range of 122-126 GHz with the shallower penetration depth are may be used to monitor the blood glucose levels.

Further, the capture device 130 may comprise one or more transmission (TX) antennas 132, one or more receiving (RX) antennas 132 and a connector 138. In one embodiment, the capture device 130 may be a wearable and portable device such as, but not limited to, a cell phone, a smartwatch, a tracker, a wearable monitor, a wristband, and a personal blood monitoring device. The one or more TX antennas 132 and the one or more RX antennas 140 may be fabricated on a substrate (not shown) within the capture device 130 in a suitable configuration. In one exemplary embodiment, at least two TX antennas 132 and at least four RX antennas 140 are fabricated on the substrate. The one or more TX antennas 132 and the one or more RX antennas 140 may correspond to a circuitry arrangement (not shown) on the substrate. Further, embodiments may include a plurality of TX antennas 132 and a plurality of RX antennas 140. The one or more TX antennas 132 and the one or more RX antennas 140 may be integrated into the circuitry arrangement. The one or more TX antennas 132 may be configured to transmit the Activated RF range signals at a pre-defined frequency. In one embodiment, the pre-defined frequency may correspond to a range suitable for the human body. For example, the one or more TX antennas 132 transmit an Activated RF range signal between 500 MHz and 300 GHz. Successively, the one or more RX antennas 140 may be configured to receive the responded portion of the Activated RF range signals. In one embodiment, the Activated RF range signals may be transmitted into the user, and electromagnetic energy may be responded from many parts such as fibrous tissue, muscle, tendons, bones, and the skin. It can be noted that effective monitoring of the blood glucose level is facilitated by an electrical response of blood molecules, such as pancreatic endocrine hormones, against the transmitted Activated RF range signals. It will be apparent to a skilled person that the pancreatic endocrine hormones such as insulin and glucagon are responsible for maintaining sugar or glucose level. Further, the electromagnetic energy responded from the blood molecules may be received by the one or more RX antennas 140.

Further, embodiments may include a frequency synthesizer 134 that may include elements to generate electrical signals at frequencies that are used by the TX antennas 132 and the RX antennas 140. In one embodiment, the frequency synthesizer 134 may include elements such as a crystal oscillator, a phase-locked loop (PLL), a frequency multiplier, and a combination thereof.

Further, embodiments may include an analog processing component 136 which may include elements such as mixers and filters. In one embodiment, the filters may include low-pass filters (LPFs). In one embodiment, the frequency synthesizer 134, the TX antennas 132, and the RX antennas 140 may be implemented in hardware as electronic circuits that are fabricated on the same semiconductor substrate.

Further, embodiments may include a connector 138 which may be a data cable that is designed for data transfer between the capture device 130 and the monitoring device 102 to send the analyte data to the monitoring device 102 to be displayed on the user interface 114. For example, the connector 138 may transmit electronic information from the capture device 130 to the monitoring device 102 and, in some embodiments, the monitoring device 102 to the capture device 130.

FIG. 2 illustrates an example operation of the base module 116. The process begins with the base module 116 initiating, at step 200, the glucose module 118. For example, the glucose module 118 begins by being initiated by the base module 116. The glucose module 118 sends the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. For example, the one or more TX antennas 132 transmit an Activated RF range signal between 500 MHz and 300 GHz. The glucose module 118 stores the RF transmit signal to memory 104. The glucose module 118 receives the RF signal from the capture device 130 RX antenna 140. For example, the one or more RX antennas 140 may be configured to receive the responded portion of the Activated RF range signals. The glucose module 118 converts to digital using the ADC converter 110. The glucose module 118 stores the RX converted signal data in memory 104. The glucose module 118 correlates the RF signals with ground truth data to determine the glucose numbers. The glucose module 118 stores the glucose number in the analyte database 126. The glucose module 118 then returns to the base module 116.

At step 202, the base module 116 may optionally call one or more of the motion module 142, the body temperature module 144, the ECG module 148, and/or the received noise module 152. The base module 116 may utilize a motion module 142 that includes at least one sensor from the group of an accelerometer, a gyroscope, an inertial movement sensor, or other similar sensor. The motion module 142 may have its own processor or utilize the processor 106 to calculate the user's movement. Motion from the user will change the blood volume in a given portion of their body and the blood flow rate in their circulatory system. This may cause noise, artifacts, or other errors in the real-time signals received by the RX antennas 140. The motion module 142 may compare the calculated motion to a motion threshold stored in memory 104. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 142 may flag the RF signals collected at the time stamp corresponding to the motion as potentially inaccurate. In some embodiments, the motion module 142 may compare RF signal data to motion data over time to improve the accuracy of the motion threshold. The motion module 142 may alert the nurse, doctor, or medical staff, such as with an audible beep or warning or a text message or alert to a connected mobile device. The alert would signal the nurse, doctor, or medical staff that the patient is moving too much to get an accurate measurement. The motion module 142 may update the analyte database 126 with the calculated motion of the user that corresponds with the received RF signal data. In this manner, the motion module 142 may be simplified to just collect motion data and allow the base module 116 to determine if the amount of motion calculated exceeds a threshold that would indicate the received RF signal data is too noisy to be relied upon for a blood glucose measurement.

The base module 116 may utilize the body temperature module 144 that includes at least one sensor from the group of a thermometer, a platinum resistance thermometer (PRT), a thermistor, a thermocouple, or another temperature sensor. The body temperature module 144 may have its own processor or utilize the processor 106 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 140. The body temperature module 144 may compare the measured temperature to a threshold temperature stored in memory 104. For example, the environmental temperature threshold may be set at zero degrees Celsius because low temperatures can cause a temporary narrowing of blood vessels which may increase the user's blood pressure. When the measured temperature exceeds the threshold, the body temperature module 144 may flag the RF signals collected at the time stamp corresponding to the temperature as potentially being inaccurate. In some embodiments, the body temperature module 144 may compare RF signal data to temperature data over time to improve the accuracy of the temperature threshold. The body temperature module 144 may alert the nurse, doctor, or medical staff, such as with an audible beep or warning or a text message or alert to a connected mobile device. The alert would signal to the nurse, doctor, or medical staff that the patient's body temperature, or the environmental temperature is not conducive to getting an accurate measurement. The body temperature module 144 updates the analyte database 126 with the measured user or environmental temperature that corresponds with the received RF signal data. In this manner, the body temperature module 144 may be simplified to just collect temperature data and allow the base module 116 to determine if the temperature measure exceeds a threshold that would indicate the received RF signal data is too noisy to be relied upon for a blood glucose measurement.

The base module 116 may utilize the ECG module 148 that includes at least one electrocardiogram sensor. The ECG module 148 may have its own processor or utilize the processor 106 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 140. The ECG module 148 may compare the measured cardiac data to a threshold stored in memory 104. For example, the threshold may be a pulse above 160 bpm, as the increased blood flow volume may cause too much noise in the received RF signal data to accurately measure the blood glucose. When the ECG data exceeds the threshold, the ECG module 148 may flag the RF signals collected at the time stamp corresponding to the ECG data as potentially being inaccurate. In some embodiments, the ECG module 148 may compare RF signal data to ECG data over time to improve the accuracy of the ECG data threshold or to improve the measurement of glucose at a given point in the cycle between peak and minimum cardiac output. The ECG module 148 may alert the nurse, doctor, or medical staff, such as with an audible beep or warning or a text message or alert to a connected mobile device. The alert would signal to the nurse, doctor, or medical staff that the patient's heart rate is not conducive to getting an accurate measurement or requires additional medical intervention. The ECG module 148 may update the analyte database 126 with the measured ECG data that corresponds with the received RF signal data. In this manner, the ECG module 148 may be simplified to just collect ECG data and allow the base module 116 to determine if the ECG data exceeded a threshold that would indicate the received RF signal data is too noisy to be relied upon for a blood glucose measurement.

The base module 116 may utilize the received noise module 152 that includes at least one sensor measuring background signals such as RF signals, Wi-Fi, and other electromagnetic signals that could interfere with the signals received by the RX antennas 140. The received noise module 152 may have its own processor or utilize the processor 106 to calculate the level of background noise being received. Background noise may interfere with or cause noise, artifacts, or other errors or inaccuracies in the real-time signals received by the RX antennas 140. The received noise module 152 may compare the level and type of background noise to a threshold stored in memory 104. The threshold may be in terms of field strength (volts per meter and ampere per meter) or power density (watts per square meter). For example, the threshold may be RF radiation greater than 300 μW/m2. When the background noise data exceeds the threshold, the received noise module 152 may flag the RF signals collected at the time stamp corresponding to background noise levels as potentially being inaccurate. In some embodiments, the received noise module 152 may compare RF signal data to background noise over time to improve the accuracy of the noise thresholds. The received radiation module may alert the nurse, doctor, or medical staff, such as with an audible beep or warning, a text message, or an alert to a connected mobile device. The alert would signal to the nurse, doctor, or medical staff that the current level of background noise is not conducive to getting an accurate measurement. The received noise module 152 may update the analyte database 126 with the background noise data that corresponds with the received RF signal data. In this manner, the received noise module 152 may be simplified to just collect background noise data and allow the base module 116 to determine if the measure exceeded a threshold that would indicate the received RF signal data is too noisy to be relied upon for a blood glucose measurement, or if an alternative transfer function should be used to compensate for the noise.

Returning to FIG. 1, the base module 116 initiates, at step 204, the analyte module 120. For example, the analyte module 120 begins by being initiated by the base module 116. The analyte module 120 sends the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. The analyte module 120 stores the RF transmit signal to memory 104. For example, the glucose module 118 stores the transmitted signal to memory 104. The analyte module 120 receives the RF signal from the capture device 130 RX antenna 140. For example, the one or more RX antennas 140 may be configured to receive the responded portion of the Activated RF range signals. The analyte module 120 converts to digital using the ADC converter 110. The analyte module 120 stores the RX converted signal data in memory 104. The analyte module 120 correlates the RF signals with ground truth data to determine the analyte numbers. The analyte module 120 stores the analyte levels or oxygen saturation levels in the analyte database 126. The analyte module 120 returns to the base module 116.

The base module 116 initiates, at step 206, the feedback module 122. For example, the feedback module 122 begins by being initiated by the base module 116. The feedback module 122 extracts the data entry from the analyte database 126. The feedback module 122 compares the extracted data entry from the analyte database 126 to the feedback database 128. The feedback module 122 determines if there is a recommendation. If it is determined that there is a recommendation the feedback module 122 extracts the recommendation from the feedback database 128. The feedback module 122 sends the extracted recommendation from the feedback database 128 to the display module 124. If it is determined that there is no recommendation or after the recommendation has been sent to the display module 124 the feedback module 122 returns to the base module 116. The base module 116 initiates, at step 208, the display module 124. For example, the display module 124 begins by being initiated by the base module 116. The display module 124 extracts the data entry from the analyte database 126. The display module 124 displays the data entry on the user interface 114. The display module 124 determines if a recommendation was received from the feedback module 122. If it is determined that there was a recommendation received from the feedback module 122 the display module 124 receives the recommendation from the feedback module 122. The display module 124 displays the received recommendation on the user interface 114. If it is determined that there was no recommendation received from the feedback module 122 or after the recommendation is displayed on the user interface 114, the display module 124 returns to the base module 116.

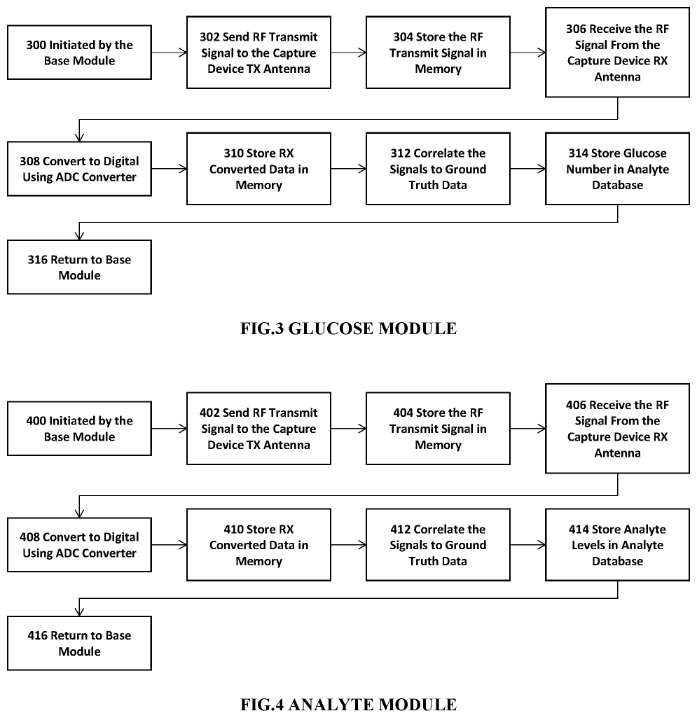

FIG. 3 illustrates an example operation of the glucose module 118. The process begins with the glucose module 118 being initiated, at step 300, by the base module 116. The glucose module 118 sends, at step 302, the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. For example, the one or more TX antennas 132 transmit an Activated RF range between 500 MHz and 300 GHz. The glucose module 118 stores, at step 304, the RF transmit signal to memory 104. For example, the glucose module 118 stores the transmitted signal to memory 104, such as an Activated RF range between 500 MHz and 300 GHz. The glucose module 118 receives, at step 306, the RF signal from the capture device 130 RX antenna 140. For example, the one or more RX antennas 140 may be configured to receive the responded portion of the Activated RF range signals. The glucose module 118 converts, at step 308, to digital using the ADC converter 110. For example, the ADC 110 may be configured to convert the received Activated RF range signals from an analog signal into a digital processor readable format. The glucose module 118 stores, at step 310, the RX converted signal data in memory 104. For example, the glucose module 118 stores the received signal from the capture device 130 RX antenna 140 that has been converted to a digital processor readable format in memory 104. The glucose module 118 correlates, at step 312, the RF signals with ground truth data to determine the glucose numbers. For example, the glucose module 118 may be configured to execute an Al correlation between the real-time ground truth data and the RX converted data. In one embodiment, the Al correlation between the real-time ground truth data and the RX-converted data is executed to determine whether the RX-converted data corresponds to the real-time ground truth data. For example, the glucose module 118 executes the Al correlation between the real-time ground truth data related to the blood glucose level of the patient as 110 mg/dL corresponding to the radio signal of frequency 122 GHz, and the 8-bit data corresponding to Activated RF range 140-155 GHz. For example, the memory 104 may store the real-time ground truth data and RX converted data. For example, the memory 104 stores the real-time ground truth data related to the blood glucose level of the patient as 110 mg/dL corresponding to the radio signal of frequency 122 GHz, and the 8-bit data corresponding to Activated RF range 140-155 GHz. The glucose module 118 stores, at step 314, the glucose number in the analyte database 126. For example, the glucose module 118 stores the patient's glucose levels in the analyte database 126 such as 220 mg/dL, 219 mg/dL, etc. The glucose module 118 then returns, at step 316, to the base module 116.

FIG. 4 illustrates an example operation of the analyte module 120. The process begins with the analyte module 120 being initiated, at step 400, by the base module 116. The analyte module 120 sends, at step 402, the RF transmit signal to the capture device 130 TX antenna 132. For example, the one or more TX antennas 132 may be configured to transmit the Activated RF range signals at a pre-defined frequency. In one embodiment, the pre-defined frequency may correspond to a range suitable for the human body. The analyte module 120 stores, at step 404, the RF transmit signal to memory 104. For example, the glucose module 118 stores the transmitted signal to memory 104. The analyte module 120 receives, at step 406, the RF signal from the capture device 130 RX antenna 140. For example, the one or more RX antennas 140 may be configured to receive the responded portion of the Activated RF range signals. The analyte module 120 converts, at step 408, to digital using the ADC converter 110. For example, the ADC 110 may be configured to convert the Activated RF range signals from an analog signal into a digital processor readable format. The analyte module 120 stores, at step 410, the RX converted signal data in memory 104. For example, the analyte module 120 stores the received signal from the capture device 130 RX antenna 140 that has been converted to a digital processor readable format in memory 104. The analyte module 120 correlates, at step 412, the RF signals with ground truth data to determine the analyte numbers. For example, the analyte module 120 may be configured to execute an Al correlation between the real-time ground truth data and the RX converted data. In one embodiment, the Al correlation between the real-time ground truth data and the RX-converted data is executed to determine whether the RX-converted data corresponds to the real-time ground truth data. For example, the analyte module 120 executes the Al correlation between the real-time ground truth data related to the analyte levels of the patient, such as oxygen saturation, lactate, cholesterol, etc. corresponding to the radio signal frequency. For example, the memory 104 may store the real-time ground truth data and RX converted data. For example, the memory 104 stores the real-time ground truth data related to the oxygen saturation level of the patient as 100% corresponding to the radio signal frequency, and the 8-bit data corresponding to the radio signal frequency range. The analyte module 120 stores, at step 414, the analyte levels or oxygen saturation levels in the analyte database 126. For example, the analyte module 120 stores the patient's oxygen saturation levels in the analyte database 126 such as 100%. In some embodiments, the analyte module 120 may store the patient's lactate levels, cholesterol levels, other analyte levels, etc. The analyte module 120 returns, at step 416, to the base module 116.

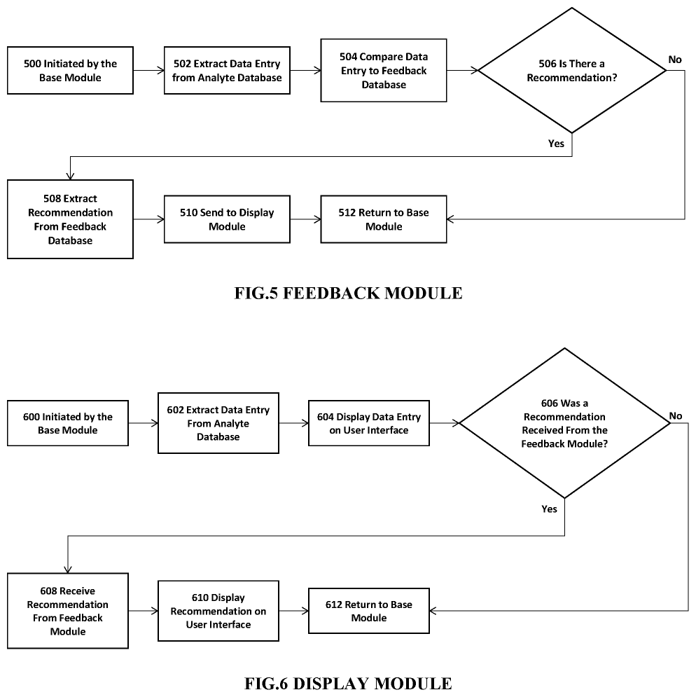

FIG. 5 illustrates an example operation of the feedback module 122. The process begins with the feedback module 122 being initiated, at step 500, by the base module 116. For example, the feedback module 122 may be initiated once the patient's real-time glucose data, oxygen saturation data, and/or other analyte data is collected. In some embodiments, the feedback module 122 may be initiated once a new data entry is added to the analyte database 126 by continuously querying the analyte database 126 for a new data entry. The feedback module 122 extracts, at step 502, the data entry from the analyte database 126. For example, the feedback module 122 extracts the new data entry from the analyte database 126, including the patient ID, the time in which the data was collected and displayed on the analyte monitoring device 102, the patient's glucose numbers and the patient's oxygen saturation levels. In some embodiments, the database may include additional analyte data, such as lactate, cholesterol, etc. The feedback module 122 compares, at step 504, the extracted data entry from the analyte database 126 to the feedback database 128. For example, the feedback module 122 may compare the patient's real-time glucose data, oxygen saturation data, and other analyte data to the feedback database 128 to determine if any of the patient's physiological parameters exceed any of the thresholds stored in the feedback database 128. In some embodiments, the feedback module 122 may input the data from the analyte database 126 into an algorithm to determine if there are any alerts, notifications, recommendations, etc. for the medical professional. For example, the algorithm may determine if any of the patient's physiological parameters exceed predetermined thresholds that would prompt any alerts, notifications, recommendations, etc. for the medical professional. The feedback module 122 determines, at step 506, if there is a recommendation. For example, if the patient's physiological parameters exceed any of thresholds stored in the feedback database 128, the corresponding rule may be to notify the medical professionals with a recommendation that would be displayed on the analyte monitoring device 102 user interface 114. For example, if the patient's glucose levels decrease and the oxygen saturation levels decrease past the thresholds the corresponding rule may be to provide the patient with dextrose and a warning may be provided to the medical professional that the patient is experiencing low blood sugar, such as hypoglycemia. For example, if the patient's oxygen saturation levels decrease the rule may be to provide the patient with oxygen. For example, if the patient's glucose levels decrease, the oxygen saturation levels decrease and the patient's blood pressure increase past the thresholds the corresponding rule may be to provide the patient with dextrose and a warning may be provided to the medical professional that the patient is experiencing low blood sugar, such as hypoglycemia. For example, if the patient's oxygen saturation levels decrease and glucose levels are in between 100 mg/dL and 130 mg/dL the rule may be to provide the patient with oxygen. For example, the feedback module 122 may extract a recommendation based upon the patient's oxygen saturation levels, glucose numbers, and blood pressure. For example, oxygen saturation is correlated with glucose levels in which glucose and arterial pressure variability are associated with markers of obstructive sleep apnea syndrome, or OSAS, severity among nondiabetic patients with lower oxygen saturation levels. Glucose and arterial pressure variability in OSAS may be used as an indicator of future diabetes in nondiabetic patients and increased cardiovascular risk. The recommendation that would be extracted would be that the patient has an increased risk of diabetes and cardiovascular risks. If it is determined that there is a recommendation the feedback module 122 extracts, at step 508, the recommendation from the feedback database 128. For example, the feedback module 122 may extract the rule, such as to notify the medical professional to provide the patient with insulin to lower the patient's glucose levels, to provide the patient with dextrose and a warning may be provided to the medical professional that the patient is experiencing low blood sugar, be to provide the patient with oxygen, etc. The feedback module 122 sends, at step 510, the extracted recommendation from the feedback database 128 to the display module 124. For example, the feedback module 122 sends the extracted rule, recommendation, notification, etc. to the display module 124. If it is determined that there is no recommendation or after the recommendation has been sent to the display module 124, the feedback module 122 returns, at step 512, to the base module 116.

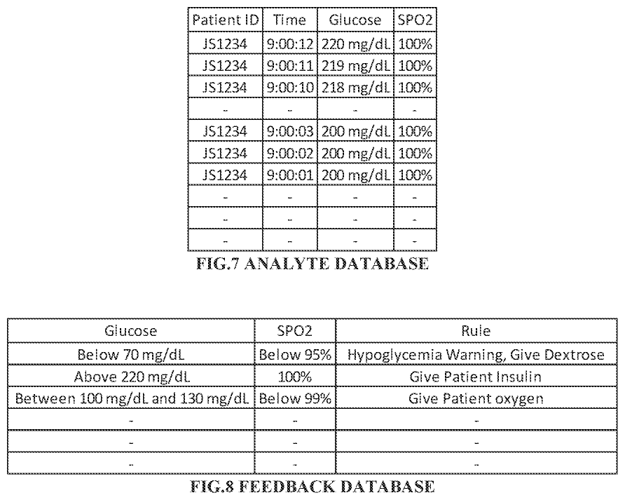

FIG. 6 illustrates an example operation of the display module 124. The process begins with the display module 124 being initiated, at step 600, by the base module 116. For example, the display module 124 may be initiated once the feedback module 122 has been completed. In some embodiments, the display module 124 may be initiated once there is a new data entry stored in the analyte database 126 to display the patient's physiological parameters. The display module 124 extracts, at step 602, the data entry from the analyte database 126. For example, the display module 124 extracts the new data entry from the analyte database 126, including the patient ID, the time in which the data was collected and displayed on the analyte monitoring device 102, the patient's glucose numbers, and the patient's oxygen saturation levels. In some embodiments, the database may include other analyte data, such as lactate levels, cholesterol levels, etc. The display module 124 displays, at step 604, the data entry on the user interface 114. For example, the display module 124 displays the new data entry from the analyte database 126 on the user interface 114, including the patient ID, the time in which the data was collected and displayed on the analyte monitoring device 102, the patient's glucose numbers, and the patient's oxygen saturation levels. In some embodiments, the data may be displayed on the user interface 114 as an individual number, shown as a graph over time, etc. to inform the medical professionals of the patient's current physiological parameters. In some embodiments, the analyte monitoring device 102 may be connected to the patient's electronic health records through a cloud connection and store the patient's physiological parameters in their electronic health record. The display module 124 determines, at step 606, if a recommendation was received from the feedback module 122. For example, the display module 124 may determine if a recommendation, rule, notification, etc. was received from the feedback module 122. If it is determined that there was a recommendation received from the fuse module 122 the display module 124 receives, at step 608, the recommendation from the feedback module 122. For example, the display module 122 may receive a recommendation, rule, notification, etc. from the feedback module 122 such as, to notify the medical professional to provide the patient with insulin to lower the patient's glucose levels, to provide the patient with dextrose and a warning may be provided to the medical professional that the patient is experiencing low blood sugar, be to provide the patient with oxygen, etc. The display module 124 displays, at step 610, the received recommendation on the user interface 114. For example, the display module 124 displays the received recommendation, rule, notification, etc. on the user interface 114 such as, to notify the medical professional to provide the patient with insulin to lower the patient's glucose levels, to provide the patient with dextrose and a warning may be provided to the medical professional that the patient is experiencing low blood sugar, be to provide the patient with oxygen, to provide the patient with medication to lower their blood pressure, etc. If it is determined that there was no recommendation received from the feedback module 122 or after the recommendation is displayed on the user interface 114, the display module 124 returns, at step 612, to the base module 116.

FIG. 7 illustrates an example of the analyte database 126. The database 126 is created from the process described for the glucose module 118 and analyte module 120 which collects data from the capture device 130. The data is compared to the feedback database 128 to determine if there is a relationship between the data that requires the medical professional's attention and provides the medical professional with a notification. The data is extracted from database 126 and displayed on the analyte monitoring device 102 user interface 114 to inform the medical professional of the patient's physiological parameters. The database 126 contains a patient ID, the time in which the data was collected and displayed on the analyte monitoring device 102, the patient's glucose numbers, and the patient's oxygen saturation levels. In some embodiments, the database may include additional analyte data, such as lactate, cholesterol, etc.

FIG. 8 illustrates an example of the feedback database 128. The database 128 contains a predetermined set of rules that provide a medical professional with a notification or alert on the analyte monitoring device 102 if the patient's physiological parameters are at certain levels or exceed certain thresholds that may cause harm to the patient. The database 128 is used in the process described for the feedback module 122 in which the data from the analyte database 126 is compared to the thresholds provided in the feedback database 128 to determine if the patient's physiological parameters exceed one, two, or a combination of the thresholds and, if so, the rule or recommendation is extracted and is displayed on the analyte monitoring device 102 user interface 114. The database 128 may contain thresholds for the patient's glucose numbers, oxygen saturation levels, and the corresponding rule. In some embodiments, the database 128 may include additional analyte data, such as lactate, cholesterol, etc. The database 128 contains a predetermined set of rules that provide a medical professional with a notification or alert on the analyte monitoring device 102 if the patient's physiological parameters are at certain levels or exceed certain thresholds that may cause harm to the patient. The database 128 is used in the process described for the feedback module 122 in which the data from the analyte database 126 is compared to the thresholds provided in the feedback database 128 to determine if the patient's physiological parameters exceed one, two, or a combination of the thresholds and, if so, the rule or recommendation is extracted and is displayed on the analyte monitoring device 102 user interface 114. The database 128 may contain thresholds for the patient's glucose numbers, oxygen saturation levels, and the corresponding rule. In some embodiments, the database 128 may include additional analyte data, such as lactate, cholesterol, etc. For example, if the patient's glucose levels decrease, the oxygen saturation levels decrease and the patient's blood pressure increase past the thresholds the corresponding rule may be to provide the patient with dextrose and a warning may be provided to the medical professional that the patient is experiencing low blood sugar, such as hypoglycemia. For example, if the patient's oxygen saturation levels decrease and glucose levels are in between 100 mg/dL and 130 mg/dL the rule may be to provide the patient with oxygen. For example, the feedback module 122 may extract a recommendation based upon the patient's oxygen saturation levels, glucose numbers, and blood pressure. For example, oxygen saturation is correlated with glucose levels in which glucose and arterial pressure variability are associated with markers of obstructive sleep apnea syndrome, or OSAS, severity among nondiabetic patients with lower oxygen saturation levels. Glucose and arterial pressure variability in OSAS may be used as an indicator of future diabetes in nondiabetic patients and increased cardiovascular risk. The recommendation that would be extracted would be that the patient has an increased risk of diabetes and cardiovascular risks.

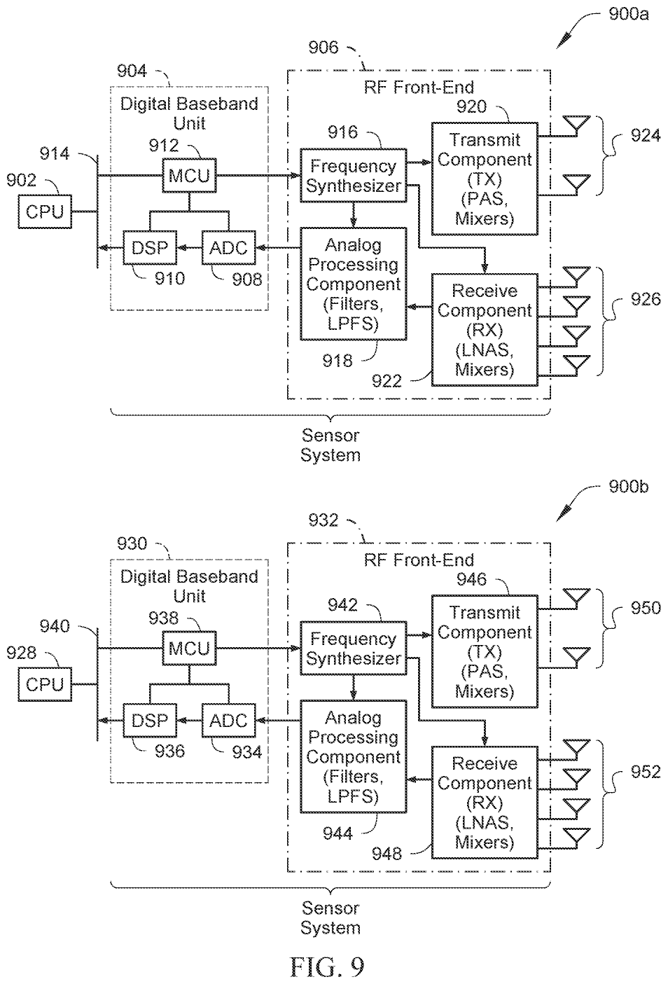

FIG. 9 illustrates an embodiment of a functional block diagram of the system utilizing Activated RF range signals to monitor the analyte levels in the user. FIG. 9 illustrates a functional block diagram of a sensor system 900a of the analyte monitoring device 102 utilizing the Activated RF range signals to monitor the blood glucose level in the user, according to an embodiment. The sensor system 900a may comprise a central processing unit (CPU) 902, a digital baseband unit 904, and a radio frequency (RF) front end 906. Further, the digital baseband unit 904 may comprise an analog-to-digital converter (ADC) 908, a digital signal processor (DSP) 910, and a microcontroller unit (MCU) 912. In one embodiment, the digital baseband unit 904 may include some other configurations, including some other combination of elements. The digital baseband unit 904 may be connected to the CPU 902 using bus connectors 914. Further, the RF front-end 906 may comprise a frequency synthesizer 916, an analog processing component 918, a transmit (TX) component 920, and a receive (RX) component 922. Further, the TX component 920 may include PAS elements. The PAS elements correspond to power, amplifiers, and mixers. The RX component 922 may include LNAS elements. The LNAS elements correspond to low noise amplifiers (LNAs), variable gain amplifiers (VGAs), and mixers. The frequency synthesizer 916 may include elements to generate electrical signals at frequencies that are used by the TX component 920 and the RX components 922. In one embodiment, the frequency synthesizer 916 may include elements such as a crystal oscillator, a phase-locked loop (PLL), a frequency multiplier, and a combination thereof. The analog processing component 918 may include elements such as mixers and filters. In one embodiment, the filters may include low-pass filters (LPFs). In one embodiment, the frequency synthesizer 916, the analog processing component 918, the TX component 920, and the RX component 922 of the RF front end 906 may be implemented in hardware as electronic circuits that are fabricated on the same semiconductor substrate. Further, the TX component 920 may comprise at least two TX antennas 924, and the RX component 922 may comprise at least four RX antennas 926. In one embodiment, the sensor system 900a may be provided with multiple TX antennas and RX antennas in a ratio of 1:2. Further, the at least two TX antennas 924 and the at least four RX antennas 926 may be configured to transmit and receive Activated RF range signals. In one embodiment, the sensor system 900a, including the CPU 902, the digital baseband unit 904, and the RF front end 906 of the analyte monitoring device 102, may be integrated into various configurations according to the size and shape of the analyte monitoring device 102. For example, some configurations of components of the analyte monitoring device 102 are fabricated on a semiconductor substrate and/or included in a packaged IC device or a combination of packaged IC devices. In one embodiment, the analyte monitoring device 102 is designed to transmit and receive Activated RF range signals at a pre-defined frequency. In one embodiment, the pre-defined frequency ranges between 122-126 GHz for Activated RF range signals.

FIG. 9 illustrates a functional block diagram of a sensor system 900b of the analyte monitoring device 102 utilizing the Activated RF range signals to monitor the analyte levels in the user, according to an embodiment. The analyte monitoring device 102 may contain separate sensor systems 900a, 900b to collect glucose data and other analyte data, such as lactate levels, cholesterol, etc. or oxygen saturation data. The sensor system 900b may comprise a central processing unit (CPU) 928, a digital baseband unit 930, and a radio frequency (RF) front end 932. Further, the digital baseband unit 930 may comprise an analog-to-digital converter (ADC) 934, a digital signal processor (DSP) 936, and a microcontroller unit (MCU) 938. In one embodiment, the digital baseband unit 930 may include some other configurations, including some other combination of elements. The digital baseband unit 930 may be connected to the CPU 928 using bus connectors 940. Further, the RF front-end 932 may comprise a frequency synthesizer 942, an analog processing component 944, a transmit (TX) component 946, and a receive (RX) component 948. Further, the TX component 946 may include PAS elements. The PAS elements correspond to power, amplifiers, and mixers. The RX component 948 may include LNAS elements. The LNAS elements correspond to low noise amplifiers (LNAs), variable gain amplifiers (VGAs), and mixers. The frequency synthesizer 942 may include elements to generate electrical signals at frequencies that are used by the TX component 946 and the RX components 948. In one embodiment, the frequency synthesizer 942 may include elements such as a crystal oscillator, a phase-locked loop (PLL), a frequency multiplier, and a combination thereof. The analog processing component 944 may include elements such as mixers and filters. In one embodiment, the filters may include low-pass filters (LPFs). In one embodiment, the frequency synthesizer 942, the analog processing component 944, the TX component 946, and the RX component 948 of the RF front end 932 may be implemented in hardware as electronic circuits that are fabricated on the same semiconductor substrate. Further, the TX component 946 may comprise at least two TX antennas 950, and the RX component 948 may comprise at least four RX antennas 952. In one embodiment, the sensor system 900b may be provided with multiple TX antennas and RX antennas in a ratio of 1:2. Further, the at least two TX antennas 950 and the at least four RX antennas 952 may be configured to transmit and receive Activated RF range signals. In one embodiment, the sensor system 900b, including the CPU 928, the digital baseband unit 930, and the RF front end 932 of the analyte monitoring device 102, may be integrated into various configurations according to the size and shape of the analyte monitoring device 102. For example, some configurations of components of the analyte monitoring device 102 are fabricated on a semiconductor substrate and/or included in a packaged IC device or a combination of packaged IC devices. In one embodiment, the analyte monitoring device 102 is designed to transmit and receive Activated RF range signals at a pre-defined frequency.

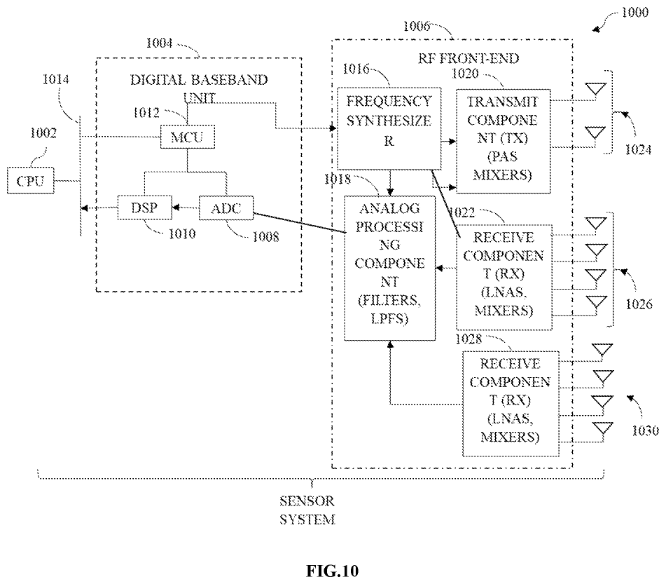

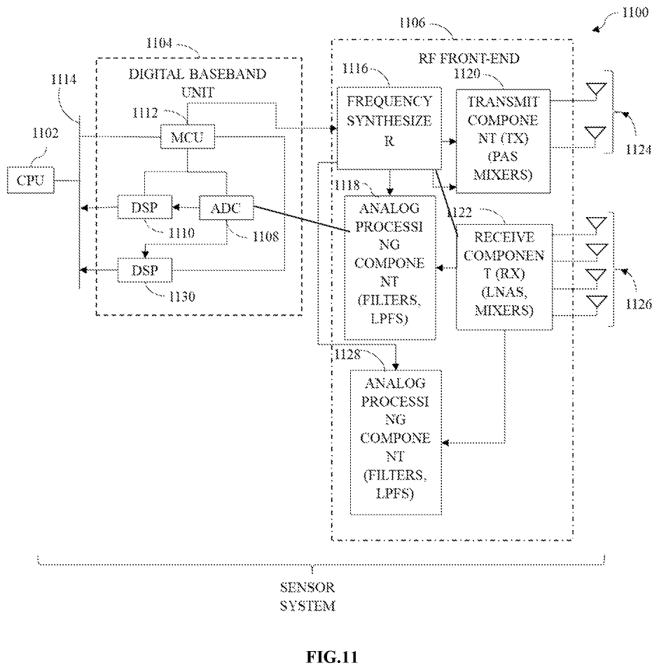

FIG. 10 illustrates another embodiment of a functional block diagram of the system utilizing Activated RF range radio waves to monitor the analyte levels in the user. The additional embodiment includes the sensor system 1000 containing a plurality of receiving components 1022, 1028 including their own RX antennas 1026, 1030. The first RX component 1022 and antennas 1026 may be used to receive resulting RF signals to detect glucose levels of the patient and the second RX component 1028 and antennas 1030 may be used to detect analyte levels or oxygen saturation levels of the patient. The signal provided to the TX component 1020 may be different depending on which RX component 1022, 1028 is used in the process. In some embodiments, the glucose module 118 may be initiated to send an initial RF signal to the TX component 1020 and the resulting signal is collected by the RX component 1022, and once the signals are cleared the analyte module 120 may be initiated to send an initial RF signal to the TX component 1020 and the resulting signal is collected by the RX component 1028. The sensor system 1000 may comprise a central processing unit (CPU) 1002, a digital baseband unit 1004, and a radio frequency (RF) front end 1006. Further, the digital baseband unit 1004 may comprise an analog-to-digital converter (ADC) 1008, a digital signal processor (DSP) 1010, and a microcontroller unit (MCU) 1012. In one embodiment, the digital baseband unit 1004 may include some other configurations, including some other combination of elements. The digital baseband unit 1004 may be connected to the CPU 1002 using bus connectors 1014. Further, the RF front-end 1006 may comprise a frequency synthesizer 1016, an analog processing component 1018, the transmit (TX) component 1020, and the plurality of receive (RX) components 1022, 1028. Further, the TX component 1020 may include PAS elements. The PAS elements correspond to power, amplifiers, and mixers. The plurality of RX components 1022, 1028 may include LNAS elements. The LNAS elements correspond to low noise amplifiers (LNAs), variable gain amplifiers (VGAs), and mixers. The frequency synthesizer 1016 may include elements to generate electrical signals at frequencies that are used by the TX component 1020 and the plurality of RX components 1022, 1028. In one embodiment, the frequency synthesizer 1016 may include elements such as a crystal oscillator, a phase-locked loop (PLL), a frequency multiplier, and a combination thereof. The analog processing component 1018 may include elements such as mixers and filters. In one embodiment, the filters may include low-pass filters (LPFs). In one embodiment, the frequency synthesizer 1016, the analog processing component 1018, the TX component 1020, and the plurality of RX components 1022, 1028 of the RF front end 1006 may be implemented in hardware as electronic circuits that are fabricated on the same semiconductor substrate. Further, the TX component 1020 may comprise the at least two TX antennas 1024, and each of the plurality of RX components 1022, 1028 may comprise the at least four RX antennas 1026, 1030. In one embodiment, the sensor system 1000 may be provided with multiple TX antennas and RX antennas in a ratio of 1:2. Further, the at least two TX antennas 1024 and the at least four RX antennas 1026, 1030 may be configured to transmit and receive Activated RF range signals. In one embodiment, the sensor system 1000, including the CPU 1002, the digital baseband unit 1004, and the RF front end 1006 of the analyte monitoring device 102, may be integrated into various configurations according to the size and shape of the analyte monitoring device 102. For example, some configurations of components of the analyte monitoring device 102 are fabricated on a semiconductor substrate and/or included in a packaged IC device or a combination of packaged IC devices. In one embodiment, the analyte monitoring device 102 is designed to transmit and receive Activated RF range signals at a pre-defined frequency. In one embodiment, the pre-defined frequency ranges between 122-126 GHz for Activated RF range signals.

FIG. 11 illustrates another embodiment of a functional block diagram of the system utilizing Activated RF range radio waves to monitor the analyte levels in the user. The additional embodiment may include a plurality of analog processing components 1118, 1128 and digital signal processors 1110, 1130. The first analog processing components 1118 may include elements designed for glucose levels, such as mixers and filters. In one embodiment, the filters may include low-pass filters (LPFs). The second analog processing components 1128 may include elements designed for analyte levels, such as mixers and filters. In one embodiment, the filters may include low-pass filters (LPFs). In some embodiments, the microcontroller 1112 may determine if the glucose module 118 or analyte module 120 sent the RF signal to determine which DSP 1110, 1130 and analog processing component 1118, 1128 to use during the process. The sensor system 1100 may comprise a central processing unit (CPU) 1102, a digital baseband unit 1104, and a radio frequency (RF) front end 1106. Further, the digital baseband unit 1104 may comprise an analog-to-digital converter (ADC) 1108, the plurality of digital signal processors (DSP) 1110, 1130, and a microcontroller unit (MCU) 1112. In one embodiment, the digital baseband unit 1104 may include some other configurations, including some other combination of elements. The digital baseband unit 1104 may be connected to the CPU 1102 using bus connectors 1114. Further, the RF front-end 1106 may comprise a frequency synthesizer 1116, the plurality of analog processing components 1118, 1128, a transmit (TX) component 1120, and a receive (RX) component 1122. Further, the TX component 1120 may include PAS elements. The PAS elements correspond to power, amplifiers, and mixers. The RX component 1122 may include LNAS elements. The LNAS elements correspond to low noise amplifiers (LNAs), variable gain amplifiers (VGAs), and mixers. The frequency synthesizer 1116 may include elements to generate electrical signals at frequencies that are used by the TX component 1120 and the RX component 1122. In one embodiment, the frequency synthesizer 1116 may include elements such as a crystal oscillator, a phase-locked loop (PLL), a frequency multiplier, and a combination thereof. The plurality of analog processing components 1118, 1128 may include elements such as mixers and filters. In one embodiment, the filters may include low-pass filters (LPFs). In one embodiment, the frequency synthesizer 1116, the plurality of analog processing components 1118, 1128, the TX component 1120, and the RX component 1122 of the RF front end 1106 may be implemented in hardware as electronic circuits that are fabricated on the same semiconductor substrate. Further, the TX component 1120 may comprise at least two TX antennas 1124, and the RX component 1122 may comprise at least four RX antennas 1126. In one embodiment, the sensor system 1100 may be provided with multiple TX antennas and RX antennas in a ratio of 1:2. Further, the at least two TX antennas 1124 and the at least four RX antennas 1126 may be configured to transmit and receive Activated RF range signals. In one embodiment, the sensor system 1100, including the CPU 1102, the digital baseband unit 1104, and the RF front end 1106 of the analyte monitoring device 102, may be integrated into various configurations according to the size and shape of the analyte monitoring device 102. For example, some configurations of components of the analyte monitoring device 102 are fabricated on a semiconductor substrate and/or included in a packaged IC device or a combination of packaged IC devices. In one embodiment, the analyte monitoring device 102 is designed to transmit and receive Activated RF range signals at a pre-defined frequency. In one embodiment, the pre-defined frequency ranges between 122-126 GHz for Activated RF range signals.

Functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1-8. (canceled)

9. A surgical care system, comprising:
a non-invasive radio frequency glucose sensor that is configured to non-invasively obtain glucose readings during surgical care of a patient using radio frequency signals, the non-invasive radio frequency glucose sensor includes one or more antennas configured to transmit radio frequency glucose detection signals into the patient and one or more antennas configured to detect radio frequency glucose signals that result from the radio frequency glucose detection signals transmitted into the patient;
a non-invasive radio frequency non-glucose analyte sensor that is configured to non-invasively obtain non-glucose analyte readings during the surgical care of the patient using radio frequency signals, the non-invasive radio frequency non-glucose analyte sensor includes one or more antennas configured to transmit radio frequency analyte detection signals into the patient and one or more antennas configured to detect radio frequency analyte signals that result from the radio frequency analyte detection signals transmitted into the patient;
an analyte database in communication with the non-invasive radio frequency glucose sensor and the non-invasive radio frequency non-glucose analyte sensor, the analyte database includes a plurality of separate time entries, each time entry includes an associated glucose reading and an associated non-glucose analyte reading;
a feedback database in communication with the analyte database, the feedback database includes a plurality of separate data entries, each data entry includes a predetermined rule and a glucose threshold and a non-glucose analyte threshold that correspond to the predetermined rule;
a monitoring device in communication with the analyte database and the feedback database during the surgical care of the patient, the monitoring device including a display that simultaneously displays, during the surgical care of the patient, an obtained glucose reading and an obtained non-glucose analyte reading and that displays one of the predetermined rules stored in the feedback database if the obtained glucose reading exceeds the corresponding glucose threshold and if the obtained non-glucose analyte reading exceeds the corresponding non-glucose analyte threshold;
an analog-to-digital converter coupled to one or more of the non-invasive radio frequency non-glucose analyte sensor or the non-invasive radio frequency non-glucose analyte sensor, the analog-to-digital converter being configured to convert analog signals into digital signals; and
a first connector that physically connects the non-invasive radio frequency glucose sensor to the monitoring device during the surgical care of the patient.

10-11. (canceled)

12. The surgical care system of claim 9, wherein the first connector comprises a cable.

13. The surgical care system of claim 9, further comprising:
a second connector that physically connects the non-invasive radio frequency non-glucose analyte sensor to the monitoring device during the surgical care of the patient.

14. The surgical care system of claim 13, wherein the second connector comprises a cable.

15. (canceled)

16. The surgical care system of claim 9, further comprising:
a second connector that physically connects the non-invasive radio frequency non-glucose analyte sensor to the monitoring device during the surgical care of the patient, wherein the first connector comprises a cable, and the second connector comprises a cable.

17. The surgical care system of claim 9, further comprising a digital baseband, the digital baseband in communication with the non-invasive radio frequency glucose sensor and the non-invasive radio frequency non-glucose analyte sensor during the surgical care of the patient.

18. The surgical care system of claim 9, further comprising a first digital baseband in communication with the non-invasive radio frequency glucose sensor during the surgical care of the patient and a second digital baseband in communication with the non-invasive radio frequency non-glucose analyte sensor during the surgical care of the patient.

19. The surgical care system of claim 9, further comprising:
a sensor configured to obtain non-analyte readings during the surgical care of the patient,
the sensor is in communication with the analyte database during the surgical care of the patient, each time entry includes an associated non-analyte reading obtained by the sensor during the surgical care of the patient,
the monitoring device being configured to determine, during the surgical care of the patient and for each time entry, based on the associated non-analyte reading, that at least one of the associated glucose reading and the associated non-glucose analyte reading are unreliable.

20. The surgical care system of claim 19, wherein the monitoring device is configured to make the determination during the surgical care of the patient based on a determination that the associated non-analyte reading exceeds a non-analyte reading threshold.

21. The surgical care system of claim 20, wherein the associated non-analyte reading comprises a body temperature reading of the patient, and wherein the determination that the associated non-analyte reading exceeds the non-analyte reading threshold is made in response to the body temperature reading being below a temperature threshold.

22. The surgical care system of claim 9, wherein the non-glucose analyte readings comprise oxygen saturation levels.

23. The surgical care system of claim 19, wherein the associated non-analyte reading comprises a patient motion reading during the surgical care of the patient, a body temperature reading of the patient during the surgical care of the patient, an electrocardiogram reading of the patient during the surgical care of the patient, or a background noise reading during the surgical care of the patient.

24. The surgical care system of claim 9, further comprising:
a capture device that includes both the non-invasive radio frequency glucose sensor and the non-invasive radio frequency non-glucose analyte sensor.

25. The surgical care system of claim 9, wherein the non-glucose analyte readings comprise lactate levels or cholesterol levels.
